SEQUENCE LISTING (8 sequences)

SEQ ID NO: 1 - DNA, 1074 bp, Artificial sequence
  Description of Artificial Sequence artificially synthesized sequence
atgatcccacccgcacgtgactcgcttatgtttgtctttgcgactgcggtaatcgctagttttttcggtagcgcaaagacactacgcggctcttccccacagcaattcgacgtatactggaatgtgccaactttcatgtgtcacaagcacggtatgaaatttgaagagctgaaagatttcggtatacaccagaacgcaatggatatgtttcgcggcgaagaaattgcgattctttatgaccctggcatgtttcccgcccttctagtagataaagatggatacgtcactaaacggaacggtggggtgccgcaagaggggaacctcaaggaacatttagaaacctttagaaagcatctgacgacacaaattccggacgagagctttagcgggataggaatcatagactttgagagttggagaccgattttcaggcagaactgggcgtcactcgagccctataaaactttgtccataaagttggagagggaaaaacacccattttggtcggaggcagctgtgaagaaggaggccaaacgtcgattcgagaaatccgggcgaatatttatggaggaaaccctcaaaatggccaaaaaactaagaccgaaagcaaagtggggctattatgggtatccccactgtttcaatcaaacccctggacagcagtcggttcactgcaatcggcaaacgatgatggaaaatgacggtatgagttggctgtttacactcgaagacgttcatgctcccagcgtttacctacgattggaaatcaaggaggatgaccggccttcattcgtgaaaggccgggtttccgaggccttaaggttagccgctaaatcgtcttcaaaacaacgtatcctaccttactactggttcatttatcaggataagaaggatgagttcttaacggaaaaggatacacaaaacactataaatatgatcgctaatctgggatctaatggtttcataatttggggatctagtgacgatgtcaacacggaacgcaaatgcaaggatttacagcaatacgtaaaggaagtcttgggcccagcgattaagaagttcacccttcattga

SEQ ID NO: 2 - protein, 357 aa, Artificial sequence
  Description of Artificial Sequence artificially synthesized sequence
MIPPARDSLMFVFATAVIASFFGSAKTLRGSSPQQFDVYWNVPTFMCHKHGMKFEELKDFGIHQNAMDMFRGEEIAILYDPGMFPALLVDKDGYVTKRNGGVPQEGNLKEHLETFRKHLTTQIPDESFSGIGIIDFESWRPIFRQNWASLEPYKTLSIKLEREKHPFWSEAAVKKEAKRRFEKSGRIFMEETLKMAKKLRPKAKWGYYGYPHCFNQTPGQQSVHCNRQTMMENDGMSWLFTLEDVHAPSVYLRLEIKEDDRPSFVKGRVSEALRLAAKSSSKQRILPYYWFIYQDKKDEFLTEKDTQNTINMIANLGSNGFIIWGSSDDVNTERKCKDLQQYVKEVLGPAIKKFTLH

SEQ ID NO: 3 - DNA, 72 bp, Artificial sequence
  Description of Artificial Sequence artificially synthesized sequence
atcccacccgcacgtgactcgcttatgtttgtctttgcgactgcggtaatcgctagttttttcggtagcgca

SEQ ID NO: 4 - DNA, 1020 bp, Artificial sequence
  Description of Artificial Sequence artificially synthesized sequence
atgaagacactacgcggctcttccccacagcaattcgacgtatactggaatgtgccaactttcatgtgtcacaagcacggtatgaaatttgaagagctgaaagatttcggtatacaccagaacgcaatggatatgtttcgcggcgaagaaattgcgattctttatgaccctggcatgtttcccgcccttctagtagataaagatggatacgtcactaaacggaacggtggggtgccgcaagaggggaacctcaaggaacatttagaaacctttagaaagcatctgacgacacaaattccggacgagagctttagcgggataggaatcatagactttgagagttggagaccgattttcaggcagaactgggcgtcactcgagccctataaaactttgtccataaagttggagagggaaaaacacccattttggtcggaggcagctgtgaagaaggaggccaaacgtcgattcgagaaatccgggcgaatatttatggaggaaaccctcaaaatggccaaaaaactaagaccgaaagcaaagtggggctattatgggtatccccactgtttcaatcaaacccctggacagcagtcggttcactgcaatcggcaaacgatgatggaaaatgacggtatgagttggctgtttacactcgaagacgttcatgctcccagcgtttacctacgattggaaatcaaggaggatgaccggccttcattcgtgaaaggccgggtttccgaggccttaaggttagccgctaaatcgtcttcaaaacaacgtatcctaccttactactggttcatttatcaggataagaaggatgagttcttaacggaaaaggatacacaaaacactataaatatgatcgctaatctgggatctaatggtttcataatttggggatctagtgacgatgtcaacacggaacgcaaatgcaaggatttacagcaatacgtaaaggaagtcttgggcccagcgattaagaagttcacccttcatcaccaccatcatcatcattga

SEQ ID NO: 5 - protein, 339 aa, Artificial sequence
  Description of Artificial Sequence artificially synthesized sequence
MKTLRGSSPQQFDVYWNVPTFMCHKHGMKFEELKDFGIHQNAMDMFRGEEIAILYDPGMFPALLVDKDGYVTKRNGGVPQEGNLKEHLETFRKHLTTQIPDESFSGIGIIDFESWRPIFRQNWASLEPYKTLSIKLEREKHPFWSEAAVKKEAKRRFEKSGRIFMEETLKMAKKLRPKAKWGYYGYPHCFNQTPGQQSVHCNRQTMMENDGMSWLFTLEDVHAPSVYLRLEIKEDDRPSFVKGRVSEALRLAAKSSSKQRILPYYWFIYQDKKDEFLTEKDTQNTINMIANLGSNGFIIWGSSDDVNTERKCKDLQQYVKEVLGPAIKKFTLHHHHHHH

SEQ ID NO: 6 - DNA, 29 bp, Artificial sequence
  Description of Artificial Sequence artificially synthesized sequence
ccggaattcatgaagacactacgcggctc

SEQ ID NO: 7 - DNA, 51 bp, Artificial sequence
  Description of Artificial Sequence artificially synthesized sequence
atttgcggccgctcaatgatgatgatggtggtgatgaagggtgaacttctt

SEQ ID NO: 8 - DNA, 1074 bp, Artificial Sequence
  Description of Artificial Sequence artificially synthesized sequence
atgataccaccagctcgagactctctgatgtttgtcttcgcgactgctgtgatcgccagtttcttcggcagtgcaaagaccctcagaggaagcagtccacagcagtttgacgtttattggaacgtgcccactttcatgtgccacaagcacggcatgaaatttgaggaactcaaagattttggaattcatcagaatgccatggacatgttccgtggtgaagagatcgcgattctttacgatcccggaatgtttccggcgttactggtcgataaggacggatacgtgacgaaaaggaacggcggcgtgccgcaagaaggaaacctgaaggagcatctcgaaacattccggaagcatttaaccactcagattcccgacgaatcgttcagcggcattggcataatagacttcgagagttggcggcctatcttccgacaaaattgggcctctctcgagccctacaagacgctgtccataaaactagaacgtgagaagcatcccttctggagcgaggcggccgtcaagaaggaagcgaagcgtcgtttcgagaagtccggcaggattttcatggaggagactctgaagatggcgaagaagctgcgacctaaggccaagtggggttactacggctatccgcattgcttcaatcagacccccggccaacaatccgtgcattgcaatcgtcagaccatgatggagaatgacggaatgtcttggctgttcacgctcgaggacgtgcacgcgccttccgtgtacttgagactggagatcaaggaggacgatcggccgagcttcgtgaagggcagagtgtcggaggcgctgcggctggcggcgaagagctcgagcaaacaacggatcttaccttattactggttcatataccaagataaaaaagatgaattcctcaccgagaaagacacgcagaacacgatcaacatgatagccaacctcggcagcaacggttttatcatttggggtagctccgatgacgtcaacaccgagcggaagtgcaaggatctacaacagtacgtgaaagaagtcctggggccggcgataaagaaattcaccctgcattaa

(19) United States
(12) Patent Application Publication
(10) Pub. No.: US 2024/0150742 A1
(43) Pub. Date: May 9, 2024

(54) GENE FOR EFFICIENTLY EXPRESSING HYALURONIC ACID HYDROLASE AND EXPRESSION METHOD THEREOF

(71) Applicant: BLOOMAGE BIOTECHNOLOGY CORPORATION LIMITED, Shandong (CN)

(72) Inventors: Hao WANG, Shandong (CN); Tianmeng ZHANG, Shandong (CN); Rongrong XU, Shandong (CN); Youheng ZHANG, Shandong (CN); Xueping GUO, Shandong (CN); Jingkun HAO, Shandong (CN)

(73) Assignee: BLOOMAGE BIOTECHNOLOGY CORPORATION LIMITED, Shandong (CN)

(21) Appl. No.: 18/280,403

(22) PCT Filed: Mar. 23, 2021
(86) PCT No.: PCT/CN2021/082346

§ 371 Date: Sep. 5, 2023

(30) Foreign Application Priority Data
Mar. 5, 2021 (CN) 202110246672.0

(51) Int. Cl. C12N 9/26; C12N 1/16; C12R 1/84

(52) U.S. Cl. CPC C12N 9/2474; C12N 1/165; C12N 2500/30; C12N 2523/00; C12R 2001/84

(57) ABSTRACT

Provided is a gene for efficiently expressing hyaluronic acid hydrolase. A nucleotide sequence of the gene is represented by SEQ ID NO: 4. By truncating a segment of a signal peptide sequence at the N-terminus of a full-length hyaluronic acid hydrolase gene, the Pichia pastoris engineering strain of high level expression genetic engineering hyaluronic acid hydrolase is constructed. The enzyme activity of the hyaluronic acid hydrolase in a fermentation broth obtained by high-density fermentation using the constructed Pichia pastoris engineering strain is up to 4.7×10⁵ U/mL.

### TECHNICAL FIELD

The application belongs to the technical field of genetic engineering, and in particular, relates to a gene for efficiently expressing hyaluronic acid hydrolase and an expression method thereof.

### BACKGROUND ART

Hyaluronidase (HAase) is a general term for a class of enzymes that can act on the β-1,3 or β-1,4 glycosidic bond of hyaluronic acid sugar chains to degrade hyaluronic acid, and is widely distributed in nature. Hyaluronidase may be widely applied in clinic as a pharmacologically active substance. For example, it is used in combination with local anesthetics in ophthalmic surgery to speed up the onset of anesthetics. It can promote the diffusion of medicine liquid, exudate or blood stored locally in the eye and the absorption of ocular vitreous opacity, prevent eyeball adhesion after conjunctival chemical burns, and eliminate related inflammatory reactions. Hyaluronic acid filling is an important means of plastic surgery, but hyaluronic acid complications may occur. Hyaluronic acid hydrolase can effectively treat the above adverse reactions as the most effective substance that may be used to hydrolyze hyaluronic acid. Hyaluronidase combined with anti-tumor drugs to assist subcutaneous administration, instead of venipuncture, to improve the medical experience of patients, is currently a hot market application with great potential. In addition, as a tool enzyme, hyaluronidase can efficiently prepare low-molecular-weight hyaluronic acid and hyaluronic acid oligosaccharides, which are more easily absorbed and utilized by the body, and also has huge market prospects in the field of food and cosmetics.

HAases are divided into three types according to differences in their origin, catalytic mechanism and substrate specificity. The first type is hyaluronic acidlyase derived from microorganisms. This type of HAase (EC 4.2.2.1) cleaves the β-1,4glycosidic bond of hyaluronic acid through the β racemization reaction, with the main product being an unsaturated disaccharide molecule 2-acetamido-2-deoxy-3-O-(β-D-gluco-4-enepyranosyluronic acid)-D-glucose. Such hyaluronic acid lyases are mainly derived from Clostridium, Micrococcus, Streptococcus, Bacillus and Streptomyces. The second type of representative HAase (EC 3.2.1.36) is mainly derived from leech salivary glands and duodenal worms, belongs to endo-β-glucuronidase, and can hydrolyze the β-1,3glycosidic bond of hyaluronic acid with the final product being mainly saturated hyaluronic acid tetrasaccharide and containing a small amount of hyaluronic acid hexasaccharide. This type of HAase has strong substrate specificity and basically does not have hydrolytic and transglycosidic activities on chondroitin sulfate and dermatan sulfate structures. The third type of hyaluronidase is endo-β-N-acetylglucosaminidase (EC 3.2.1.35), is mainly derived from mammalian testis and animal venom, and can hydrolyze β-1,4glycosidic bond of hyaluronic acid with the final product being mainly hyaluronic acid tetrasaccharide. Such enzymes have a broad substrate spectrum, also have certain catalytic activity on chondroitin sulfate and dermatan sulfate structures, and have transglycosidic activity.

The research on the first type of hyaluronic acidlyase and the second type of endo-β-glucuronidase has been reported in many literatures. Although there are also reports on the third type of endo-β-N-acetylglucosaminidase, the reports mainly focus on the PH20 enzyme derived from mammalian testis, and there are few reports on hyaluronidase of animal venom. In 2017, Kohei Kazuma et al. conducted a complete analysis of the venom components of odontomachusmonticola for the first time based on transcriptome and peptidomics techniques, and identified a full-length hyaluronic acid hydrolase sequence. At present, there is no relevant report on the recombinant expression of hyaluronic acid hydrolase in odontomachusmonticola.

### SUMMARY

In view of the problems existing in the prior art, the present application provides a gene for efficiently expressing hyaluronic acid hydrolase and an expression method thereof.

Specifically, the present application relates to the following aspects:
1. A gene for efficiently expressing hyaluronic acid hydrolase, having a nucleotide sequence as shown in SEQ ID NO.4.
2. A protein encoded by the gene of item 1, having a sequence as shown in SEQ ID NO.5.
3. A recombinant expression vector comprising the nucleotide sequence of item 1.
4. The recombinant expression vector of item 3, wherein a plasmid backbone is the Pichia pastoris vector of pPIC series or pGAP series or pAO815, preferably pPIC9K.
5. A Pichia pastoris, comprising the expression vector of item 3 or 4.
6. A method for producing hyaluronic acid hydrolase, comprising:
producing the hyaluronic acid hydrolase by using a recombinant Pichia pastoris strain comprising the gene of item 1 or the Pichia pastoris of item 5.
7. The method of item 6, wherein the Pichia pastoris is a strain of GS115, KM71 or SMD1168.
8. The method of item 6, wherein the method comprises producing the hyaluronic acid hydrolase through fermentation by BMMY medium and methanol induction.
9. The method of item 8, wherein conditions of the fermentation are as follows: fermentation temperature is 25° C.-30° C., and 0.5%-1% (v/v) methanol is supplemented every 24 hours during fermentation process to induce expression for 96 hours.
10. The method of item 6, wherein the method comprises producing the hyaluronic acid hydrolase through fermentation by BSM medium and methanol induction.
11. The method of item 10, wherein the method comprises steps of:
inoculating Pichia pastoris into a seed medium to obtain a seed liquid;
inoculating the seed liquid into a BSM fermentation medium for fermentation to obtain a fermentation broth containing the hyaluronic acid hydrolase, wherein a fermentation process sequentially comprise four stages: initial fermentation, feeding, starvation culture, and methanol induction.
12. The method of item 11, wherein conditions of the initial fermentation are as follows: fermentation temperature is 25° C.-30° C., and pH is 5-7.
13. The method of item 11, wherein a time of starvation culture is 2-3 hours.
14. The method of item 11, wherein the methanol induction is performed at a temperature of 25° C.-30° C. for 80-120 hours.
15. The method of item 11, wherein the method further comprises purifying the fermentation broth.
16. The method of item 15, wherein the purification is performed by affinity chromatography using nickel column media and gradient elution using 100-500 mM imidazole buffer.
17. Use of the protein of item 2 in the preparation of hyaluronic acid-containing adjuvants, foods and cosmetics.

In the present application, a Pichia pastoris engineering strain expressing a high level of genetically engineered hyaluronic acid hydrolase is constructed by truncating a segment of a signal peptide sequence at the N-terminus of the full-length hyaluronic acid hydrolase gene. The enzymatic activity of hyaluronic acid hydrolase in the fermentation broth obtained by high-density fermentation using the constructed Pichia pastoris engineering strain is up to 4.7×10⁵ U/mL. Therefore, it will be beneficial to further reduce the cost of industrial production of the hyaluronic acid hydrolase of odontomachusmonticola, thereby expanding the application scope of the hyaluronidase in the fields of medicine, chemical industry and food.

### BRIEF DESCRIPTION OF THE DRAWINGS

FIG. 1 shows a nucleotide sequence comparison of the codon-optimized hyaluronic acid hydrolase gene and the wild-type gene.

FIG. 2 shows the SDS-PAGE graph of the fermentation supernatant of recombinant hyaluronic acid hydrolase, wherein lane M represents the standard protein with a molecular weight of 180 kDa; lane 1 represents the fermentation supernatant of recombinant strain P. pastoris GS115/pPIC9K (negative control); lane 2 represents the fermentation supernatant of codon-optimized recombinant strain P. pastoris GS115/pPIC9K-HYAL_OM^{opt} (positive control); and lane 3 represents the fermentation supernatant of the recombinant strain P. pastoris GS115/pPIC9K-ΔN24HYAL_OM^{opt} constructed in the present application.

FIG. 3 shows the crude enzymatic activity of the fermentation supernatant of the codon-optimized recombinant strain P. pastoris GS115/pPIC9K-HYAL_OM^{opt} and the recombinant strain P. pastoris GS115/pPIC9K-ΔN24HYAL_OM^{opt} constructed in the present application as determined by DNS method.

FIG. 4 shows the biomass and enzymatic activity curves of recombinant strain P. pastoris GS115/pPIC9K-ΔN24HYAL_OM^{opt} cultured at high density in a 5-L fermenter.

FIG. 5 shows the SDS-PAGE graph of the fermentation supernatant of recombinant strain P. pastoris GS115/pPIC9K-ΔN24HYAL_OM^{opt} after gradient elution with imidazole buffer of different concentrations, wherein lane M represents the standard protein with a molecular weight of 180 kDa; and lanes 1-4 represent imidazole concentrations of 100 mM, 200 mM, 300 mM, and 500 mM, respectively.

### DETAILED DESCRIPTION

The present application will be further described below in conjunction with the examples, and it should be understood that the examples are only used to further illustrate and explain the present application, and are not intended to limit the present application.

Unless otherwise defined, related technical and scientific terms in the present description have the same meaning as those commonly understood by one of ordinary skill in the art. Although methods and materials similar or identical to those described herein may be used in experiments or practical applications, the materials and methods are described below. In case of conflict, the present description, including definitions therein, shall prevail. In addition, the materials, methods and examples are illustrative and not restrictive. The present application is further described below in conjunction with specific examples, but is not intended to limit the scope of the present application.

As used herein, the term “gene” or “coding sequence” refers to a nucleotide sequence in vitro or in vivo that encodes a gene product. In some instances, the gene consists of or essentially consists of coding sequence, that is, a sequence that encodes the gene product. In other instances, the gene comprises additional, non-coding, sequence. For example, the gene may or may not include regions preceding and following the coding region, e.g., 5′ untranslated (5′ UTR) or “leader” sequences and 3′ UTR or “trailer” sequences, as well as intervening sequences (introns) between individual coding segments (exons).

As used herein, the terms “polypeptide”, “peptide”, and “protein” are used interchangeably herein to mean a polymer of amino acid residues. That is, the description for the polypeptide is also applicable to the peptide and protein, and vice versa. The terms apply to a natural amino acid polymer and an amino acid polymer in which one or more amino acid residues are non-naturally encoded amino acids. As used herein, the terms encompass amino acid chains of any length,

As used herein, the term “polynucleotide” or “nucleotide” means a single-stranded or double-stranded deoxyribonucleotide, deoxyribonucleoside, ribonucleoside or ribonucleotide and polymers thereof. Unless specifically limited, the terms cover nucleic acids containing known analogs of natural nucleotides that have binding properties similar to reference nucleic acids and are metabolized in a manner similar to naturally occurring nucleotides. Unless otherwise limited specifically, the terms also mean oligonucleotide analogs, including PNAs (peptide nucleic acids), DNA analogs used in antisense techniques (phosphorothioate, phosphoramidate, etc.), etc. Unless otherwise specified, a particular nucleic acid sequence also implicitly encompasses its conserved modified variants (including, but not limited to, degenerate codon substitutions) and complementary sequences as well as explicitly specified sequences.

As used herein, the term “vector” is used to describe a nucleic acid molecule that may be engineered to contain a cloned polynucleotide or polynucleotides that may be amplified in a host cell. Vectors include, but are not limited to, nucleic acid molecules that are single-stranded, double-stranded, or partially double-stranded; nucleic acid molecules that comprise one or more free ends, or no free ends (e.g. circular); nucleic acid molecules that comprise DNA, RNA, or both; and other varieties of polynucleotides known in the art. One type of the vector is a “plasmid”, which refers to a circular double-stranded DNA loop into which additional DNA segments can be inserted, such as by standard molecular cloning techniques. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operably linked. Such vectors are referred to herein as “expression vectors.” Recombinant expression vectors can comprise a nucleic acid of the present application in a form suitable for expression of the nucleic acid in a host cell, which means that the recombinant expression vectors include one or more regulatory elements, which may be selected on basis of the host cells to be used for expression, that is operably linked to the nucleic acid sequence to be expressed.

As used herein, codon optimization refers to a process of modifying a nucleic acid sequence for enhanced expression in the host cells by replacing at least one codon of the natural sequence with codons that are more frequently or most frequently used in the genes of that host cell while maintaining the natural amino acid sequence. Various species exhibit particular bias for certain codons of a particular amino acid. Codon bias (differences in codon usage between organisms) often correlates with the efficiency of translation of messenger RNA (mRNA), which is in turn believed to be especially dependent on the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

Codon optimization can be achieved, for example, by transforming nucleotide sequences of one species into the genetic sequence of a different species. Optimal codons help to achieve faster translation rates and high accuracy. As a result of these factors, translational selection is expected to be stronger in highly-expressed genes. However, while optimal codon usage is contemplated herein for expression of disclosed proteins, all possible codons are contemplated for use herein for nucleic acids encoding any disclosed protein.

As used herein, the term “signal peptide” means a short peptide (usually 16-30 amino acids) present at the N-terminus of the majority of newly synthesized proteins that are destined to enter the secretory pathway. It may also be referred to as signal sequence, targeting signal, localization signal, localization sequence, transit peptide, leader sequence or leader peptide. The signal peptide is normally cleaved off from the protein by a signal peptidase.

Specifically, firstly, for the full-length gene sequence of hyaluronic acid hydrolase of odontomachusmonticola, the codon optimization is performed according to the codon preference of Pichia pastoris to obtain a codon-optimized hyaluronic acid hydrolase gene, the sequence of which is shown in SEQ ID NO. 1. The protein sequence encoded by it is shown in SEQ ID NO. 2, which is consistent with the amino acid sequence encoded by the hyaluronic acid hydrolase gene of the wild-type odontomachusmonticola. Further, a segment of a signal peptide sequence (SEQ ID NO.3) is truncated at the N-terminus of the codon-optimized hyaluronic acid hydrolase gene to obtain the gene for highly expressing hyaluronic acid hydrolase of the present application, the sequence of which is shown in SEQ ID NO.4, and the protein sequence encoded by it is shown in SEQ ID NO.5. Removing the nucleotide sequence shown in SEQ ID NO.3 from the N-terminus of the codon-optimized hyaluronic acid hydrolase gene as shown in SEQ ID NO.1 does not cause translation frameshift, hyaluronic acid hydrolase can be expressed and the expression level and/or enzymatic activity of the recombinant hyaluronic acid hydrolase can be significantly improved. The method for truncating the signal peptide at the N-terminus may be any method known in the art.

The hyaluronic acid hydrolase of odontomachusmonticola belongs to the third type of hyaluronidase which is endo-β-N-acetylglucosaminidase, and can hydrolyze the β-1,4glycosidic bond of hyaluronic acid to produce the final product having a reducing end of N-acetylglucosamine. In addition, this type of enzyme has a wider substrate spectrum compared with the second type of hyaluronic acid hydrolase of leech, and also has a certain catalytic activity on the structures of chondroitin, chondroitin sulfate and dermatan sulfate. The hyaluronic acid hydrolase of odontomachusmonticola can enrich the types of hyaluronic acid hydrolysates, and may have a wider range of applications than hyaluronidase catalysis.

Pichia pastoris is a type of yeast in methylotrophic yeast that can utilize methanol as the sole carbon source and energy source. Like other yeasts, it mainly exists in the form of haploid in the asexual growth phase. When the environmental nutrition is limited, two zygotic haploid cells with different physiological types are often induced to mate and fuse into a diploid. Another biological feature of Pichia pastoris is that the alcohol oxidase required for methanol metabolism is sorted into the peroxisome, resulting in regionalization. When glucose is used as carbon source, there are only one or a few small peroxisomes in the thalluses, while when methanol is used as carbon source, peroxisomes account for almost 80% of the entire cell volume, and AOX increases to 35%-40% of total cell protein. Therefore, when the foreign protein gene is inserted by homologous recombination in front of the AOX gene, a large amount of expression can be obtained. At the same time, according to the characteristics of methanol yeast that can form peroxisomes, this system may be used to express some toxic proteins and easily degraded enzymes, and may also be used to study the biogenesis of cell-specific regionalization and its mechanism and function, providing inspiration for similar studies in higher animals.

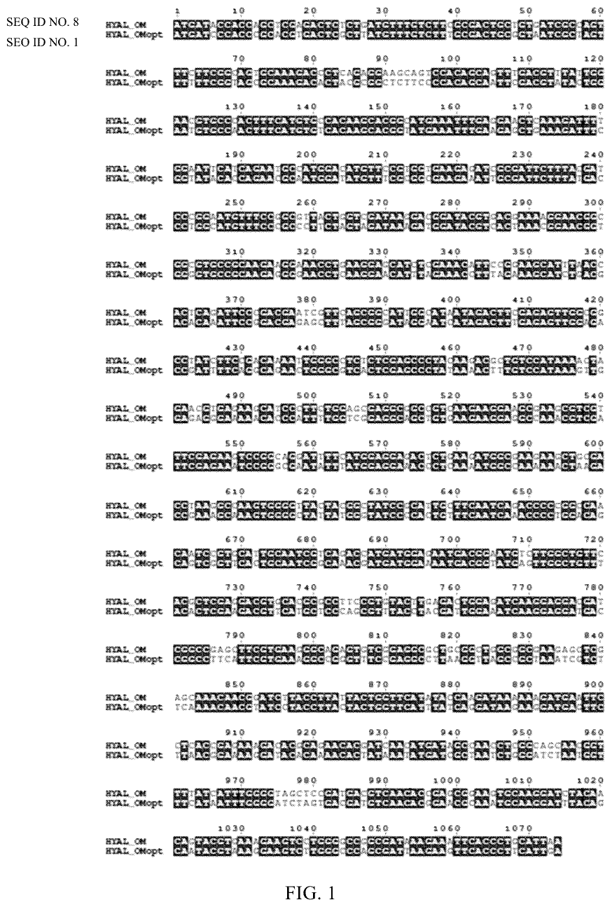

As shown in FIG. 1, the sequence homology between the optimized hyaluronic acid hydrolase gene of odontomachusmonticola and the wild-type gene is 73.2%.

The present application also provides a recombinant expression vector comprising the nucleotide sequence of the present application. Further, the backbone of the recombinant expression vector is the Pichia pastoris vector of pPIC series or pGAP series or pAO815, preferably the vector pPIC9K. The Pichia pastoris vector pPIC series includes pPIC9K, pPIC3.5K, pPICZαA,B,C, pPIC9K-His, pPICZA,B,C, etc, with the alcohol oxidase gene promoter PAOX1 as the promoter. The Pichia pastoris vector of pGAP series includes pGAPZαA, pGAPZαB, pGAPZαC, pGAPZA, pGAPZB, pGAPZC, etc., with the glyceraldehyde-3-phosphate dehydrogenase promoter P_{GAP} as the promoter. The vector pPIC9K is a Pichia pastoris protein expression vector. The kana resistance gene exists in the pPIC9K vector, which allows the use of kana resistance to screen polyclonal copies in yeast. The rest of the vector is exactly the same as the pPIC9 vector. The host bacteria of Pichia pastoris that may be used with the pPIC9K vector include a strain of KM71, GS115 or SMD1168.

The present application also provides a Pichia pastoris comprising the above-mentioned expression vector, that is, an expression vector comprising the nucleotide sequence as shown in SEQ ID NO.4.

The present application also provides a method for expressing the hyaluronic acid hydrolase gene of the present application, comprising the step of:
producing the hyaluronic acid hydrolase by using a recombinant Pichia pastoris strain containing the hyaluronic acid hydrolase gene of the present application or the Pichia pastoris provided by the present application.

In a specific embodiment, the method comprises producing the hyaluronic acid hydrolase through fermentation by BMMY medium and methanol induction.

The composition of the BMMY medium is yeast extract 10 g/L, peptone 20 g/L, K₂HPO₄ 3 g/L, KH₂PO₄ 11.8 g/L, YNB 3.4 g/L, ammonium sulfate 10 g/L, biotin 4×10⁻⁴ g/L, and methanol 10 mL/L.

In a preferred embodiment, conditions of the fermentation are as follows: the fermentation temperature is 25° C.-30° C., and 0.5%-1% (v/v) methanol is supplemented every 24 hours during fermentation process to induce expression for 96 hours.

In a more preferred embodiment, the recombinant Pichia pastoris P. pastoris GS115/pPIC9K-ΔN24HYAL_OM^{opt} containing the hyaluronic acid hydrolase gene of the present application is used as a production strain to produce the hyaluronic acid hydrolase by fermentation. The specific fermentation conditions are as follows:
P. pastoris GS115/pPIC9K-ΔN24HYAL_OM^{opt} is inoculated in 40 mL of YPD medium, cultured at 30° C. for 24 hours, and then the seed culture solution is inoculated into 40 mL of BMGY medium at 10% of the inoculum amount for 24 hours of culture. The thalluses are centrifuged, washed, and transferred to the induction medium BMMY, and 1% (v/v) methanol is supplemented every 24 hours to induce expression for 96 hours.

The present application also provides another method for expressing the hyaluronic acid hydrolase gene of the present application, comprising the following steps:
Pichia pastoris is inoculated into a seed medium to obtain a seed liquid;
the seed liquid is inoculated into the BSM fermentation medium for fermentation to obtain a fermentation broth containing hyaluronic acid hydrolase, wherein the fermentation process sequentially includes four stages: initial fermentation, feeding, starvation culture, and methanol induction.

The composition of BSM medium is glycerol 40 g/L, K₂SO₄ 18 g/L, KOH 4.13 g/L, 85% H₃PO₄ 26.7 mL/L, CaSO₄·2H₂O 0.93 g/L, MgSO₄·7H₂O 14.9 g/L, and filter-sterilized PTM14.4 mL/L. The formula of PTM1 is: CuSO₄·5H₂O 6 g/L, KI 0.09 g/L, MnSO₄·H₂O 3 g/L, H₃BO₃ 0.02 g/L, MoNa₂O₄·2H₂O 0.2 g/L, CoCl₂·6H₂O 0.92 g/L, ZnCl₂ 20 g/L, FeSO₄·7H₂O 65 g/L, biotin 0.2 g/L, and H₂SO₄ 5.0 mL.

In the first stage of initial fermentation, the fermentation temperature is 25° C.-30° C., and the pH is 5-7. The pH is controlled by automatic addition of ammonia water.

In the second stage of feeding, after the glycerol in the BSM medium is exhausted, the batch feeding stage is entered, and 50% (v/v) glycerol (containing 12 mL/L PTM1) is supplemented by exponential feeding, while the rotation speed is set to couple with dissolved oxygen DO. In a preferred embodiment, the feeding rate is 13.5 mL/h/L, 16.2 mL/h/L, 19.2 mL/h/L, 22.8 mL/h/L, 27.2 mL/h/L and 32.4 mL/h/L respectively for the first 6 hours, and is set to 30 mL/h/L for the following 6 hours.

In the third stage of starvation culture, starvation culture is performed for 2-3 hours until the residual glycerol is exhausted, wherein the starvation culture is to study the effect of a single factor on the growth of microorganisms or the production of metabolites, the cells that have been cultured to a certain stage are separated from the medium, and cultured for a certain period of time without the nutrient matrix to exhaust endogenous nutrients accumulated in the cells.

In the fourth stage of methanol induction, the methanol induction is performed at a temperature of 25° C.-30° C. for 80-120 hours.

In a preferred embodiment, pure methanol containing 12 mL/L PTM1 is fed with the final concentration maintained at 1.8% (v/v), while the fermentation temperature is adjusted to 25° C., the rotation speed is increased to 1000 rpm, and the feeding rate of the methanol and the final concentration of the methanol in the medium are controlled online in real time by a methanol detector.

The method of the present application further includes further purifying the above-mentioned fermentation broth.

In a preferred embodiment, the hyaluronic acid hydrolase protein is purified by affinity chromatography using nickel column media and gradient elution using 100-500 mM imidazole buffer.

The enzymatic activity characterization of the hyaluronic acid hydrolase produced by fermentation is determined by the plate transparent circle method, and the enzymatic activity of the fermentation broth supernatant is determined by the 3,5-dinitrosalicylic acid method (DNS).

The definition of activity unit (U) of hyaluronic acid hydrolase is as follows: the amount of enzyme required to release 1 μg glucose reducing equivalent of reducing sugar from hyaluronic acid sugar chain per hour at pH 5.5 and 38° C.

DNS reducing sugar assay: the hyaluronic acid hydrolase of odontomachusmonticola hydrolyzes the β-1,4glycosidic bond of hyaluronic acid to produce a reducing sugar product with a hydroxyl group at the reducing end. The amount of reducing sugar product relative to glucose reducing equivalents produced by hydrolysis of hyaluronic acid is measured by the DNS reducing sugar method, to calculate the catalytic activity of hyaluronic acid hydrolase.

The present application also provides use of the protein as shown in SEQ ID NO. 5 in the preparation of hyaluronic acid-containing adjuvants, foods and cosmetics.

### EXAMPLE

### Example 1 Construction of an Expression System Containing Codon-Optimized Hyaluronic Acid Hydrolase Gene (HYAL_OMopt)

Based on the full-length gene sequence of hyaluronic acid hydrolase of odontomachusmonticola (Genbank: FX985505.1) published in the NCBI database, the codon optimization was carried out according to the codon preference of Pichia pastoris. As shown in FIG. 1, the optimized gene sequence is shown in SEQ ID NO. 1, and has 73.2% homology with the sequence of wild-type gene. The amino acid sequence encoded by the optimized gene is shown in SEQ ID NO. 2, which is consistent with the amino acid sequence encoded by the wild-type gene. The codon-optimized sequence of hyaluronic acid hydrolase of odontomachusmonticola was entrusted to Nanjing GenScript Biotechnology Co., Ltd. for whole gene synthesis, and cloned into the expression vector of Pichia pastoris pPIC9K between the EcoRI and NotI enzyme cutting sites to obtain recombinant expression Vector pPIC9K-HYAL_OM^{opt}. The recombinant sequence was correct after DNA sequencing and comparison. The recombinant expression plasmid pPIC9K-HYAL_OM^{opt} was linearized by SalI fast cutting enzyme and then electro-transformed into P. pastoris GS115 expressing host cells. The recombinant transformants were screened by Geneticin G418 to obtain high-copy recombinant Pichia pastoris P. pastoris GS115/pPIC9K-HYAL_OM^{opt}.

### Example 2 Construction of an Expression System Containing the Gene for Highly Expressing Hyaluronic Acid Hydrolase (ΔN24HYAL_OM^{opt})

Using the above-mentioned pPIC9K-HYAL_OM^{opt} recombinant expression vector as a template, primers were designed. A segment of the signal peptide sequence at the N-terminus of the full-length hyaluronic acid hydrolase gene was truncated to obtain a genetically engineered hyaluronic acid hydrolase gene fragment.

The primer sequences are as follows:

Upstream primer F:
(SEQ ID NO. 6)
5′-CCGGAATTCATGAAGACACTACGCGGCTC-3′;
Downstream primer R:
(SEQ ID NO. 7)
5′-ATTTGCGGCCGCTCAATGATGATGATGGTGGT
GATGAAGGGTGAACTTCTT-3′;

It should be noted that the GAATTC sequence in the upstream primer is the introduced EcoRI restriction enzyme cutting site, the GCGGCCGC sequence in the downstream primer is the introduced NotI restriction enzyme site, and the reverse complementary sequence of the ATGATGATGATGGTGGTG sequence in the downstream primer encodes 6×His-tag tags.

The amplified genetically engineered hyaluronic acid hydrolase gene fragment was subjected to EcoRI and NotI double enzyme digestion, then cloned into the linear expression vector pPIC9K treated with the same digestion, and transformed into E. coli TOP10. The recombinant expression plasmid pPIC9K-ΔN24HYAL_OM^{opt} was obtained under the premise of no frameshifting, and the recombinant sequence was correct after DNA sequencing and comparison. The recombinant plasmid was linearized by restriction endonuclease SalI and then electro-transformed into P. pastoris GS115 cells. The recombinant transformants were screened by Geneticin G418 to obtain the recombinant strain P. pastoris GS115/pPIC9K-ΔN24HYAL_OM^{opt} with high-copy hyaluronic acid hydrolase gene.

### Example 3 Heterologous Expression of Hyaluronic Acid Hydrolase by Recombinant Pichia pastoris

The obtained recombinant engineering strain P. pastoris GS115/pPIC9K-HYAL_OM^{opt} and P. pastoris GS115/pPIC9K-ΔN24HYAL_OM^{opt} were respectively subjected to shake flask fermentation culture. The fermentation steps were as follows: a single clone was picked, inoculated in 40 mL of YPD medium (yeast extract 10 g/L, peptone 20 g/L, glucose 20 g/L), and cultured at 30° C., 200 rpm for 24 hours. It was transferred to 40 mL of initial expression medium BMGY (yeast extract 10 g/L, peptone 20 g/L, K₂HPO₄ 3 g/L, KH₂PO₄ 11.8 g/L, YNB 3.4 g/L, ammonium sulfate 10 g/L, biotin 4×10⁻⁴ g/L, glycerol 10 mL/L) at 10% inoculum amount, and cultured at 30° C., 200 rpm for 24 hours. The thalluses were collected by centrifugation, washed with normal saline, and then changed to 40 mL of induction expression medium BMMY (yeast extract 10 g/L, peptone 20 g/L, K₂HPO₄ 3 g/L, KH₂PO₄ 11.8 g/L, YNB 3.4 g/L, ammonium sulfate 10 g/L, biotin 4×10⁻⁴ g/L, methanol 10 mL/L) for culture, pure methanol was added to the medium every 24 hours to a final concentration of 1.0% (v/v) to induce expression for 96 hours.

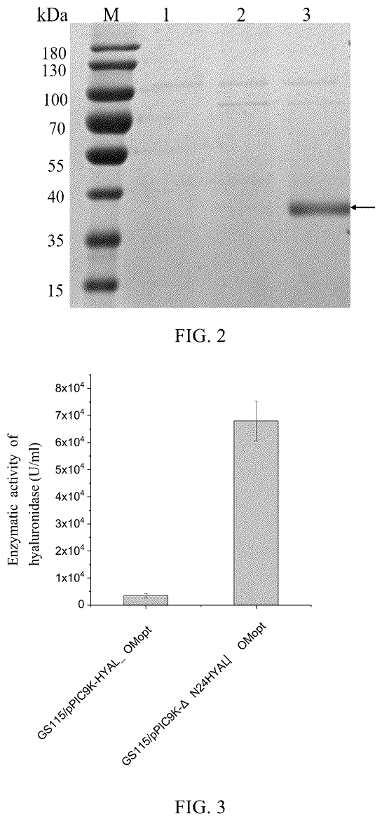

The fermentation supernatant was detected by SDS-PAGE protein electrophoresis, and the results are shown in FIG. 2. Compared with the recombinant strain in lane 2 (the recombinant strain P. pastoris GS115/pPIC9K-HYAL_OM^{opt} containing the full-length hyaluronic acid hydrolase gene sequence), there is an obvious protein band (indicated by the arrow) at the theoretical molecular weight of about 39 kDa, indicating that the recombinase has achieved efficient secretion expression after truncation of its signal peptide sequence.

### Example 4 Enzymatic Activity Detection of Shake Flask Fermentation Supernatant of the Recombinant Strain

The reducing sugar equivalent produced by the hydrolysis of hyaluronic acid was determined by DNS method, and the activity of hyaluronic acid hydrolase was calculated by standard curve with analytically pure glucose. 1 mL of reaction system: 10 μL of fermentation supernatant (the boil-inactivated fermentation supernatant with an equal volume was used as the blank) and 190 μL of 50 mM citric acid buffer (pH 5.5) were added to 800 μL of 2 mg/mL hyaluronic acid substrate solution, and incubated at 38° C. for 15 min. After the reaction, the mixture was immediately placed in a boiling water bath for 2 min to inactivate the enzyme to terminate the reaction. The treated 1 mL reaction solution was added to 2 mL DNS (potassium sodium tartrate tetrahydrate 248 g/L, 3,5-dinitrosalicylic acid 6.3 g/L, 2M NaOH 250 mL/L, phenol 5.136 g/L, sodium sulfite 5 g/L) solution. The obtained mix was shake to mix well, subjected for boiling water bath together with the glucose standard curve sample for 10 min, and then cooled to room temperature in an ice bath. 7 mL of deionized water was added and shaking was performed to mix well. The absorbance was measured at 540 nm, and converted into the amount of reducing sugar produced by the reaction. The crude enzymatic activity of the fermentation supernatant of the recombinant engineering strain was calculated according to the glucose standard curve. The results are shown in FIG. 3, the enzymatic activity of the fermentation supernatant of the recombinant strain P. pastoris GS115/pPIC9K-HYAL_OM^{opt} constructed by the present application is 3547.88 U/mL, and the enzymatic activity of the fermentation supernatant of the recombinant strain P. pastoris GS115/pPIC9K-ΔN24HYAL_OM^{opt} reaches 67970.04 U/mL.

### Example 5 High-Density Culture of Recombinant Strains in a 5-L Fermenter

The recombinant strain P. pastoris GS115/pPIC9K-ΔN24HYAL_OM^{opt} was subjected to high density culture in a 5-L fermentor. The single colony on the YPD plate was transferred to 50 mL of YPD liquid medium, cultured at 220 rpm, 30° C. for 24 hours, and the culture solution was inoculated into 200 mL of BMGY medium at 10%, and cultured at 30° C., 220 rpm for 24 hours. The 200 mL seed solution cultured for 24 hours was added into a 5-L fermenter containing 2 L BSM fermentation medium (glycerol 40 g/L, K₂SO₄ 18 g/L, KOH 4.13 g/L, 85% H₃PO₄ 26.7 mL/L, CaSO₄·2H₂O 0.93 g/L, MgSO₄·7H₂O 14.9 g/L, filter-sterilized PTM14.4 mL/L; the formula of PTM1 is: CuSO₄·5H₂O 6 g/L, KI 0.09 g/L, MnSO₄·H₂O 3 g/L, H₃BO₃ 0.02 g/L, MoNa₂O₄·2H₂O 0.2 g/L, CoCl₂·6H₂O 0.92 g/L, ZnCl₂ 20 g/L, FeSO₄·7H₂O 65 g/L, biotin 0.2 g/L, H₂SO₄ 5.0 mL). The initial fermentation parameters were set as temperature of 30° C., pH of 5.5, aeration volume of 2.0 vvm and rotation speed of 500 rpm. During the fermentation process, the pH was controlled at 5.5 by automatically adding ammonia water. After the glycerol in the BSM medium was exhausted, the batch feeding stage was entered. 50% (v/v) glycerol (containing 12 mL/L PTM1) was supplemented by exponential feeding, while the rotation speed was set to couple with dissolved oxygen DO. The feeding rate was 13.5 mL/h/L, 16.2 mL/h/L, 19.2 mL/h/L, 22.8 mL/h/L, 27.2 mL/h/L and 32.4 mL/h/L respectively for the first 6 hours, and is set to 30 mL/h/L for the following 6 hours. After the feeding, the stage of starvation culture was entered. The starvation culture was performed for 2-3 hours until the residual glycerol was exhausted. The stage of methanol induction was entered. The pure methanol containing 12 mL/L PTM1 was fed with the final concentration maintained at 1.8% (v/v), while the fermentation temperature was adjusted to 25° C., the rotation speed was increased to 1000 rpm, and the feeding rate of the methanol and the final concentration of the methanol in the medium were controlled online in real time by a methanol detector. After entering the stage of methanol induction, samples were taken at regular intervals to detect the enzymatic activity of the fermentation supernatant.

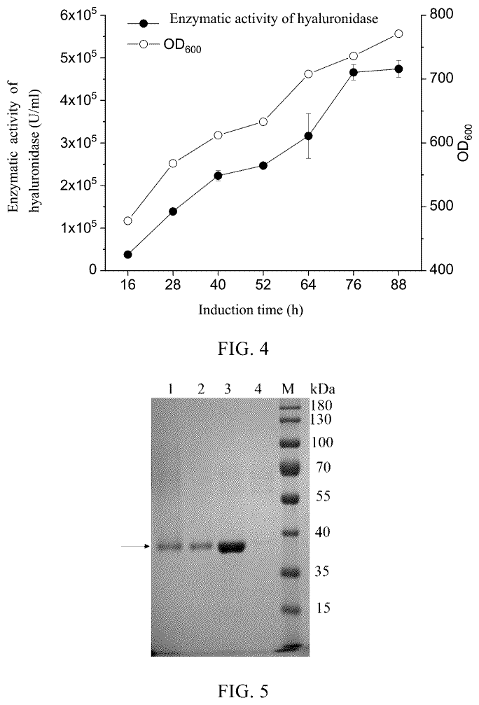

The detection results of enzymatic activity are shown in FIG. 4. When methanol induction is performed for 88 hours, the highest enzymatic activity of hyaluronic acid hydrolase in the fermentation supernatant is 4.7×10⁵ U/mL. The enzymatic activity or protein expression level obtained by high-density fermentation is 6.97 times that of shake flask fermentation, making it possible for industrial production.

### Example 6 Purification of Genetically Engineered Hyaluronic Acid Hydrolase

Six polyhistidine purification tags were introduced at the C-terminus of the genetically engineered hyaluronic acid hydrolase, and could specifically bind to nickel ions to purify the protein. Firstly, the nickel column media was filled into an affinity chromatography column, and then the nickel column was activated with 5 column volumes of binding buffer (20 mM sodium phosphate, 0.5 NaCl, 20 mM imidazole, pH 6.0). An appropriate amount of ultrafiltration concentrated fermentation supernatant filtered through 0.22 μm was added, impurities were eluted with 5 column volumes of binding buffer, and then the target protein was gradient eluted with 10 column volumes of elution buffer having 100 mM, 200 mM, 300 mM, and 500 mM imidazole, respectively. The eluent was verified by SDS-PAGE, and the results are shown in FIG. 5. Lane 3 is the protein band by elution with 300 mM imidazole buffer, with the highest protein concentration and a single band. The obtained pure protein of genetically engineered hyaluronic acid hydrolase has normal hyaluronic acid hydrolase activity by enzymatic activity assay.

Sequence listing

SEQ ID NO. 1:

| | |
|---|---|
| atgatcccacccgcacgtgactcgcttatgtttgtctttgcgactgcggtaatcgctagt | 60 |
| tttttcggtagcgcaaagacactacgcggctcttccccacagcaattcgacgtatactgg | 120 |
| aatgtgccaactttcatgtgtcacaagcacggtatgaaatttgaagagctgaaagatttc | 180 |
| ggtatacaccagaacgcaatggatatgtttcgcggcgaagaaattgcgattctttatgac | 240 |
| cctggcatgtttcccgcccttctagtagataaagatggatacgtcactaaacggaacggt | 300 |
| ggggtgccgcaagaggggaacctcaaggaacatttagaaacctttagaaagcatctgacg | 360 |
| acacaaattccggacgagagctttagcgggataggaatcatagactttgagagttggaga | 420 |
| ccgattttcaggcagaactgggcgtcactcgagccctataaaactttgtccataaagttg | 480 |
| gagagggaaaaacacccattttggtcggaggcagctgtgaagaaggaggccaaacgtcga | 540 |
| ttcgagaaatccgggcgaatatttatggaggaaaccctcaaaatggccaaaaaactaaga | 600 |
| ccgaaagcaaagtggggctattatgggtatccccactgtttcaatcaaacccctggacag | 660 |
| cagtcggttcactgcaatcggcaaacgatgatggaaaatgacggtatgagttggctgttt | 720 |
| acactcgaagacgttcatgctcccagcgtttacctacgattggaaatcaaggaggatgac | 780 |
| cggccttcattcgtgaaaggccgggtttccgaggccttaaggttagccgctaaatcgtct | 840 |
| tcaaaacaacgtatcctaccttactactggttcatttatcaggataagaaggatgagttc | 900 |
| ttaacggaaaaggatacacaaaacactataaatatgatcgctaatctgggatctaatggt | 960 |
| ttcataatttggggatctagtgacgatgtcaacacggaacgcaaatgcaaggatttacag | 1020 |
| caatacgtaaaggaagtcttgggcccagcgattaagaagttcacccttcattga | 1074 |
| SEQ ID NO. 2: | |
| Met Ile Pro Pro Ala Arg Asp SerLeu Met Phe Val Phe Ala Thr Ala | |
| 1 5 10 15 | |
| Val Ile Ala SerPhePheGlySer Ala Lys ThrLeuArgGlySerSer | |
| 20 25 30 | |
| Pro GlnGlnPhe Asp Val Tyr TrpAsn Val Pro ThrPhe Met Cys His | |
| 35 40 45 | |
| Lys His Gly Met Lys PheGluGluLeu Lys Asp PheGly Ile His Gln | |
| 50 55 60 | |
| Asn Ala Met Asp Met PheArgGlyGluGlu Ile Ala Ile Leu Tyr Asp | |
| 65 70 75 80 | |
| Pro Gly Met Phe Pro Ala LeuLeu Val Asp Lys Asp Gly Tyr Val Thr | |
| 85 90 95 | |
| Lys ArgAsnGlyGly Val Pro GlnGluGly AsnLeu Lys Glu His Leu | |
| 100 105 110 | |
| GluThrPheArg Lys His LeuThrThrGln Ile Pro Asp GluSerPhe | |
| 115 120 125 | |
| SerGly Ile Gly Ile Ile Asp PheGluSerTrpArg Pro Ile PheArg | |
| 130 135 140 | |
| GlnAsnTrp Ala SerLeuGlu Pro Tyr Lys ThrLeuSer Ile Lys Leu | |
| 145 150 155 160 | |
| GluArgGlu Lys His Pro PheTrpSerGlu Ala Ala Val Lys LysGlu | |
| 165 170 175 | |
| Ala Lys ArgArgPheGlu Lys SerGly Arg Ile Phe Met GluGluThr | |
| 180 185 190 | |
| Leu Lys Met Ala Lys LysLeuArg Pro Lys Ala Lys TrpGly Tyr Tyr | |
| 195 200 205 | |
| Gly Tyr Pro His CysPheAsnGlnThr Pro GlyGlnGlnSer Val His | |
| 210 215 220 | |
| CysAsnArgGlnThr Met MetGluAsn Asp Gly Met SerTrpLeuPhe | |
| 225 230 235 240 | |
| ThrLeuGlu Asp Val His Ala Pro Ser Val Tyr LeuArgLeuGlu Ile | |
| 245 250 255 | |
| Lys Glu Asp AspArg Pro SerPhe Val Lys Gly Arg Val SerGlu Ala | |
| 260 265 270 | |
| LeuArgLeu Ala Ala Lys SerSerSer Lys GlnArg Ile Leu Pro Tyr | |
| 275 280 285 | |
| Tyr TrpPhe Ile Tyr Gln Asp Lys Lys Asp GluPheLeuThrGlu Lys | |
| 290 295 300 | |
| Asp ThrGlnAsnThr Ile Asn Met Ile Ala AsnLeuGlySerAsnGly | |
| 305 310 315 320 | |
| Phe Ile IleTrpGlySerSer Asp Asp Val AsnThrGluArg Lys Cys | |
| 325 330 335 | |
| Lys Asp LeuGlnGln Tyr Val Lys Glu Val LeuGly Pro Ala Ile Lys | |
| 340 345 350 | |
| Lys PheThrLeu His | |
| 355 | |

SEQ ID NO. 3

| | |
|---|---|
| atcccacccgcacgtgactcgcttatgtttgtctttgcgactgcggtaatcgctagtttt | 60 |
| ttcggtagcg ca | 72 |
| SEQ ID NO. 4 | |
| atgaagacactacgcggctcttccccacagcaattcgacgtatactggaatgtgccaact | 60 |
| ttcatgtgtcacaagcacggtatgaaatttgaagagctgaaagatttcggtatacaccag | 120 |
| aacgcaatggatatgtttcgcggcgaagaaattgcgattctttatgaccctggcatgttt | 180 |
| cccgcccttctagtagataaagatggatacgtcactaaacggaacggtggggtgccgcaa | 240 |
| gaggggaacctcaaggaacatttagaaacctttagaaagcatctgacgacacaaattccg | 300 |
| gacgagagctttagcgggataggaatcatagactttgagagttggagaccgattttcagg | 360 |
| cagaactgggcgtcactcgagccctataaaactttgtccataaagttggagagggaaaaa | 420 |
| cacccattttggtcggaggcagctgtgaagaaggaggccaaacgtcgattcgagaaatcc | 480 |
| gggcgaatatttatggaggaaaccctcaaaatggccaaaaaactaagaccgaaagcaaag | 540 |
| tggggctattatgggtatccccactgtttcaatcaaacccctggacagcagtcggttcac | 600 |
| tgcaatcggcaaacgatgatggaaaatgacggtatgagttggctgtttacactcgaagac | 660 |
| gttcatgctcccagcgtttacctacgattggaaatcaaggaggatgaccggccttcattc | 720 |
| gtgaaaggccgggtttccgaggccttaaggttagccgctaaatcgtcttcaaaacaacgt | 780 |
| atcctaccttactactggttcatttatcaggataagaaggatgagttcttaacggaaaag | 840 |
| gatacacaaaacactataaatatgatcgctaatctgggatctaatggtttcataatttgg | 900 |
| ggatctagtgacgatgtcaacacggaacgcaaatgcaaggatttacagcaatacgtaaag | 960 |
| gaagtcttgggcccagcgattaagaagttcacccttcatcaccaccatcatcatcattga | 1020 |
| SEQ ID NO. 5: | |
| Met Lys ThrLeuArgGlySerSer Pro GlnGlnPhe Asp Val Tyr Trp | |
| 1 5 10 15 | |
| Asn Val Pro ThrPhe Met Cys His Lys His Gly Met Lys PheGluGlu | |
| 20 25 30 | |
| Leu Lys Asp PheGly Ile His GlnAsn Ala Met Asp Met PheArgGly | |
| 35 40 45 | |
| GluGlu Ile Ala Ile Leu Tyr Asp Pro Gly Met Phe Pro Ala LeuLeu | |
| 50 55 60 | |
| Val Asp Lys Asp Gly Tyr Val Thr Lys ArgAsnGlyGly Val Pro Gln | |
| 65 70 75 80 | |
| GluGly AsnLeu Lys Glu His LeuGluThrPheArg Lys His LeuThr | |
| 85 90 95 | |
| ThrGln Ile Pro Asp GluSerPheSerGly Ile Gly Ile Ile Asp Phe | |
| 100 105 110 | |
| GluSerTrp Arg Pro Ile PheArgGlnAsnTrp Ala SerLeuGlu Pro | |
| 115 120 125 | |
| Tyr Lys ThrLeuSer Ile Lys LeuGluArgGlu Lys His Pro PheTrp | |
| 130 135 140 | |
| SerGlu Ala Ala Val Lys LysGlu Ala Lys ArgArgPheGlu Lys Ser | |
| 145 150 155 160 | |
| Gly Arg Ile Phe Met GluGluThrLeu Lys Met Ala Lys LysLeuArg | |
| 165 170 175 | |
| Pro Lys Ala Lys TrpGly Tyr TyrGly Tyr Pro His CysPheAsnGln | |
| 180 185 190 | |
| Thr Pro GlyGlnGlnSer Val His CysAsnArgGlnThr Met MetGlu | |
| 195 200 205 | |
| Asn Asp Gly Met SerTrpLeuPheThrLeuGlu Asp Val His Ala Pro | |
| 210 215 220 | |
| Ser Val Tyr LeuArgLeuGlu Ile Lys Glu Asp AspArg Pro SerPhe | |
| 225 230 235 240 | |
| Val Lys Gly Arg Val SerGlu Ala LeuArgLeu Ala Ala Lys SerSer | |
| 245 250 255 | |
| Ser Lys GlnArg Ile Leu Pro Tyr TyrTrpPhe Ile Tyr Gln Asp Lys | |
| 260 265 270 | |
| Lys Asp GluPheLeuThrGlu Lys Asp ThrGlnAsnThr Ile Asn Met | |
| 275 280 285 | |
| Ile Ala AsnLeuGlySerAsnGly Phe Ile IleTrpGlySerSer Asp | |
| 290 295 300 | |
| Asp Val AsnThrGluArg Lys Cys Lys Asp LeuGlnGln Tyr Val Lys | |
| 305 310 315 320 | |
| Glu Val LeuGly Pro Ala Ile Lys LysPheThrLeu His HisHisHis | |
| 325 330 335 | |
| His HisHis | |
| SEQ ID NO. 6: | |
| CCGGAATTCATGAAGACACTACGCGGCTC | |
| SEQ ID NO. 7: | |
| ATTTGCGGCCGCTCAATGATGATGATGGTGGTGATGAA GGGTGAACTTCTT | |

1. A gene for efficiently expressing hyaluronic acid hydrolase, having a nucleotide sequence as shown in SEQ ID NO.4.

2. A protein encoded by the gene of claim 1, having a sequence as shown in SEQ ID NO.5.

3. A recombinant expression vector, comprising the nucleotide sequence of claim 1.

4. The recombinant expression vector of claim 3, wherein a plasmid backbone is a Pichia pastoris vector of pPIC series, or pGAP series, or pAO815, preferably pPIC9K.

5. A Pichia pastoris, comprising the expression vector of claim 3.

6. A method for producing hyaluronic acid hydrolase, comprising the step of:
producing the hyaluronic acid hydrolase by using a recombinant Pichia pastoris strain comprising the gene of claim 1.

7. The method of claim 6, wherein the Pichia pastoris is a strain of GS115, KM71 or SMD1168.

8. The method of claim 6, wherein the method comprises producing the hyaluronic acid hydrolase through fermentation by BMMY medium and methanol induction.

9. The method of claim 8, wherein conditions of the fermentation are as follows: fermentation temperature is 25° C.-30° C., and 0.5%-1% (v/v) methanol is supplemented every 24 hours during fermentation process to induce expression for 96 hours.

10. The method of claim 6, wherein the method comprises producing the hyaluronic acid hydrolase through fermentation by BSM medium and methanol induction.

11. The method of claim 10, wherein the method comprises steps of:
inoculating Pichia pastoris into a seed medium to obtain a seed liquid;
inoculating the seed liquid into a BSM fermentation medium for fermentation to obtain a fermentation broth containing the hyaluronic acid hydrolase, wherein a fermentation process sequentially comprise four stages: initial fermentation, feeding, starvation culture, and methanol induction.

12. The method of claim 11, wherein conditions of the initial fermentation are as follows: fermentation temperature is 25° C.-30° C., and pH is 5-7.

13. The method of claim 11, wherein a time of the starvation culture is 2-3 hours.

14. The method of claim 11, wherein the methanol induction is performed at a temperature of 25° C.-30° C. for 80-120 hours.

15. The method of claim 11, wherein the method further comprises purifying the fermentation broth.

16. The method of claim 15, wherein the purification is performed by affinity chromatography using nickel column media and gradient elution using 100-500 mM imidazole buffer.

17. Use of the protein of claim 2 in the preparation of hyaluronic acid-containing adjuvants, foods and cosmetics.
